(19) United States
(12) Patent Application Publication
(10) Pub. No.: US 2024/0033269 A1
(43) Pub. Date: Feb. 1, 2024

(54) PPARAXANTHINE-BASED COMPOSITIONS FOR INHIBITING INFLAMMATION, IMPROVING JOINT HEALTH, AND ENHANCING IMMUNE FUNCTION

(71) Applicant: PX ING, LLC, Lewisville, TX (US)

(72) Inventors: Ralf Jäger, Whitefish Bay, WI (US); Kylin Liao, Plano, TX (US); Shawn D. Wells, Frisco, TX (US); Martin Purpura, Spring, TX (US)

(21) Appl. No.: 18/227,808

(22) Filed: Jul. 28, 2023

Related U.S. Application Data
(60) Provisional application No. 63/393,204, filed on Jul. 28, 2022.

(51) Int. Cl. A61K 31/522; A61K 38/05; A61K 45/06; A61P 29/00

(52) U.S. Cl. CPC A61K 31/522; A61K 38/05; A61K 45/06; A61P 29/00

(57) ABSTRACT

The disclosed technology relates generally to compositions, methods, and system for utilizing paraxanthine alone and in combination for use in enhancing immune function and/or inhibiting inflammation through administration of a paraxanthine-containing composition to a subject. More particularly, the disclosure relates to paraxanthine and other compounds, whether produced synthetically or derived from natural sources, and use of these compounds to provide physiological benefits, which may vary according to paraxanthine concentration and the presence of synergists and antagonists.

### CROSS-REFERENCE TO RELATED APPLICATION(S)

This application claims priority to U.S. Provisional Application No. 63/393,204 filed Jul. 28, 2022, and entitled “PARAXANTHINE-BASED COMPOSITIONS FOR INHIBITING INFLAMMATION, IMPROVING JOINT HEALTH, AND ENHANCING IMMUNE FUNCTION,” which is hereby incorporated by reference in its entirety under 35 U.S.C. § 119(e).

### TECHNICAL FIELD

The disclosed technology relates generally to compositions, methods, and system for utilizing paraxanthine alone and in combination for use in enhancing immune function and inhibiting inflammation through administration of paraxanthine-containing composition to a subject. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Paraxanthine is also known as 1,7-dimethylxanthine or 1,7-dimethyl-3H-purine-2,6-dione. Paraxanthine is structurally related to caffeine as well as a metabolite of caffeine which is also found through caffeine excretion in humans. In humans and other animals, caffeine is first degraded to either paraxanthine, theobromine or theophylline, and then later, to a methylated xanthine.

Inflammation is a key mediator of numerous pathological conditions. Further, a well-functioning immune system is critical for the maintenance of health. There is a need in the art for compositions and methods to inhibit inflammatory processes and promote or enhance immune function.

### BRIEF SUMMARY

Disclosed herein is a nutritional supplement for improving immune function and/or inhibiting inflammation comprising from about 2 mg to about 800 mg paraxanthine, either natural or synthetic. In certain aspects, the paraxanthine is present in amount from about 50 mg to about 400 mg. In further embodiments, the paraxanthine is present in amount from about 20 mg to about 600 mg. In certain embodiments, the disclosed nutritional supplement further comprises an effective amount of dileucine.

In certain embodiments, the nutritional supplement is a dietary supplement. In exemplary implementations, the dietary supplement is powder or a capsule. In further embodiments, the nutritional supplement is a functional food. In exemplary implementations, the functional food is a beverage, nutrition bar, yoghurt, or cereal.

Disclosed herein is a method for enhancing immune function in a subject by providing the subject with a composition comprising about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

Further disclosed herein is a method of inhibiting inflammation in a subject in need thereof comprising administering to the subject a composition comprising about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain embodiments, the subject is at risk of developing inflammatory disease or condition. In further embodiments, the subject has been diagnosed with an inflammatory disease or condition. In exemplary implementations, the subject has been diagnosed with diabetes, Crohn's disease, rheumatoid arthritis, fibromyalgia, systemic lupus erythematosus, glomerulonephritis, scleroderma, or multiple sclerosis.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

As used herein, the terms “effective amount” and “amount effective” refer to an amount that is sufficient to achieve the desired result or effect (e.g., increasing immune function and/or decreasing inflammation).

As used herein, the terms “nutritional supplement” and “dietary supplement” refer to any product that is added to the diet. In some particularly preferred embodiments, nutritional supplements are taken by mouth and often contain one or more dietary ingredients, including but not limited to vitamins, minerals, herbs, amino acids, enzymes, and cultures of organisms.

The terms “modulate”, “modulating”, “modulation”, “enhance”, “enhancing”, and “enhancement” are used synonymously herein to describe the improved ability of any human or animal (including, but not limited to, a dog, cat, rodent, horse, sheep, cow, pig, goat, donkey, chicken, or rabbit) to mount an immune response.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with an inflammatory disorder” means having been subjected to a physical examination by a person of skill, for example, a physician, and found to have a condition that can be diagnosed or treated by a compound or composition that can increase immune function and/or decrease inflammation. As a further example, “diagnosed with a need for enhance immune function” refers to having been subjected to a physical examination by a person of skill, for example, a physician, and found to have a condition characterized by an infection or other disease wherein increasing immune function would be beneficial to the subject. Such a diagnosis can be in reference to an inflammatory disease or condition and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein “inflammatory disease or condition” includes, but is not limited to: which the compositions and methods herein may be used include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Disclosed herein is a nutritional supplement for improving immune function and/or inhibiting inflammation comprising from about 2 mg to about 800 mg paraxanthine, either natural or synthetic. In certain aspects, the paraxanthine is present in amount from about 50 mg to about 400 mg. In further embodiments, the paraxanthine is present in amount from about 20 mg to about 600 mg.

In certain embodiments, the disclosed nutritional supplement further comprises an effective amount of dileucine. In certain embodiments, dileucine is present in an amount of from about 200 mg to about 5000 mg.

In certain embodiments, the nutritional supplement is a dietary supplement. In exemplary implementations, the dietary supplement is powder or a capsule. In further embodiments, the nutritional supplement is a functional food. In exemplary implementations, the functional food is a beverage (e.g. a coffee beverage), nutrition bar, yoghurt, or cereal.

Disclosed herein is a method for enhancing immune function in a subject by providing the subject with a composition comprising about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain embodiments, the composition also includes dileucine. In certain embodiments, dileucine is present in an amount of from about 200 mg to about 5000 mg.

In certain embodiments, administration of the composition to the subject increases Th2, Th17, and/or Tfh levels in the subject relative to a control subject. In certain embodiments, administration of the composition to the subject increases Th2, Th17, and/or Tfh levels in the subject from about 350% to about 800% relative to a control subject. In certain embodiments, Th2 is increased by about 370%. In further embodiments, Th17 is increased by about 760%. In still further embodiments, Tfh is increased by about 600%.

In certain embodiments, administration of the composition to the subject produces an increase in serum IL-2 levels in the subject relative to a control subject. In certain embodiments, administration of the composition to the subject produces an increase in serum IL-2 levels in the subject from about 10% to about 30% relative to a control subject. In still further embodiments, IL-2 levels are increased by about 20% in the subject.

According to certain embodiments, administration of the composition to the subject produces a decrease in serum Th1 and/or iTreg levels in the subject relative to a control subject. In certain embodiments, and/or iTreg levels are decreased from about 20% to about 65%. In certain embodiments, wherein Th1 is decreased by about 30%. In further embodiments, iTreg is decreased by about 60%.

In certain embodiments, the composition also includes one or more additional actives selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.

In certain embodiments, the one or more additional active is an anti-inflammatory agent. Disclosed anti-inflammatory agents include agents that exert an anti-inflammatory effect by inhibiting prostaglandins (e.g. targeting COX-2), increasing macrophage mediated phagocytosis (e.g. Resolvins), suppressing cytokine-driven inflammation (e.g., glucocorticoids) suppressing cytokine-driven inflammation, inhibiting cytokines, inhibiting histone deacetylation, inhibiting kinases, stimulating PPAR and/or inhibiting proteases.

Anti-inflammatory agents include, but are not limited to, aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.

In further embodiments, steroidal anti-inflammatory agents, is a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilo se hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. The anti-inflammatory agent may also be a biological inhibitor of proinflammatory signaling molecules including antibodies to such biological inflammatory signaling molecules.

In certain embodiments, the paraxanthine is derived from a natural source. In further embodiments, the paraxanthine is synthetic.

According to certain embodiments, administration of the disclosed composition increases factors associated with adaptive immune function in the subject. In exemplary embodiments,

Further disclosed herein is a method of inhibiting inflammation in a subject in need thereof comprising administering to the subject a composition comprising about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain embodiments, the subject is at risk of developing inflammatory disease or condition. In further embodiments, the subject has been diagnosed with an inflammatory disease or condition. In exemplary implementations, the subject has been diagnosed with diabetes, Crohn's disease, rheumatoid arthritis, fibromyalgia, systemic lupus erythematosus, glomerulonephritis, scleroderma, or multiple sclerosis.

In still further embodiments, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

According to certain embodiments, administration of the composition to the subject produces a decrease in proinflammatory factors in the subject. In certain embodiments, administration of the composition to the subject yields a decrease in serum IL-6 levels in the subject relative to a control subject. In further embodiments, administration of the composition to the subject produces a decrease in serum IL-6 levels in the subject from about 5% to about 15% relative to a control subject. In further embodiments, administration of the composition to the subject produces a decrease in serum IL-6 levels in the subject of about 8% relative to a control subject.

According to further embodiments, administration of the composition to the subject produces a decrease in serum CRP levels in the subject relative to a control subject. In certain embodiments, administration of the composition to the subject produces a decrease in serum CRP levels in the subject from about 15% to about 30% relative to a control subject. In certain implementations, administration of the composition to the subject produces a decrease in serum CRP levels in the subject of about 18% relative to a control subject.

According to certain embodiments, administration of the composition to the subject produces an increase in anti-inflammatory factors in the subject. In certain embodiments, administration of the composition to the subject produces an increase in serum IL-10 levels in the relative to a control subject. In certain implementations, administration of the composition to the subject produces an increase in serum IL-10 levels in the subject from about 5% to about 20% relative to a control subject. In further embodiments, administration of the composition to the subject produces an increase in serum IL-10 levels in the subject of about 8% relative to a control subject.

Further disclosed herein are method for improving joint health by administering to a subject in need thereof a composition comprising paraxanthine. In certain embodiments, the composition further comprises dileucine.

According to certain embodiments, improving joint health comprises alleviating or reducing the severity of at least one symptom of osteoarthritis, such as, for example, pain, stiffness, tenderness, reduced flexibility, grating sensation, bone spurs, swelling, or any combination thereof. Thus, in some embodiments, there is provided a method of reducing the severity of at least one symptom of osteoarthritis in a subject with osteoarthritis, comprising administering to the subject an herbal composition of the present disclosure.

In some embodiments of the fourth aspect, the improvement in joint health may be measured by changes in baseline versus end point scores in the 30 second chair stand test (30SCST). Thus, in some embodiments, provided is a method of improving the 30 second chair stand test (30SCST) score in a subject with an inflammatory joint condition comprising administering to the subject an composition of the present disclosure for at least 30 days, at least 60 days, at least 90 days, at least 120 days, or longer. In some embodiments, the improvement in the 30SCST at 120 days is at least 8%, at least 10%, at least 13%, at least 15%, at least 16%, or at least about 18%.

In some embodiments of the fourth aspect, the improvement in joint health may be measured by changes in baseline versus end point range of motion in knee flexion. Thus, in some embodiments, there is provided a method of improving the range of motion in knee flexion in a subject with an inflammatory joint condition comprising administering to the subject a composition of the present disclosure for at least 30 days, at least 60 days, at least 90 days, at least 120 days, or longer. In some embodiments, the range of motion in knee flexion at 120 days is improved by at least 4%, at least 5%, at least 6%, or at least 7%.

As described herein, an effective amount of the composition can be administered to the subject once per day. In some embodiments, an effective amount of the composition or can be administered to a subject as multiple doses per day, for example twice per day or more frequently, three times per day or more frequently, or four times per day or more frequently. In some embodiments, an effective amount of the composition can be administered to a subject once per week or more frequently, twice per week or more frequently, three times per week or more frequently, four times per week or more frequently, five times per week or more frequently, or six times per week or more frequently.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present disclosure are defined by the following numbered clauses:
1. A method for enhancing immune function in a subject, comprising:
   providing the subject with a composition comprising about 2 mg to about 800 mg of paraxanthine.
2. The method of clause 1, wherein paraxanthine is present in the composition in amount from about 20 mg to about 600 mg.
3. The method of clause 2, wherein paraxanthine is present in the composition in amount from about 50 mg to about 400 mg.
4. The method of any of clauses 1-3, wherein the composition further comprises an effective amount of dileucine.
5. The method of any of clauses 1-3, wherein the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.
6. The method of any preceding clause wherein the paraxanthine is derived from a natural source.
7. The method of any preceding clause wherein the paraxanthine is synthetic.
8. The method of any preceding clause, wherein administration of the composition to the subject increases Th2, Th17, and/or Tfh levels in the subject relative to a control subject.
9. The method of clause 8, wherein administration of the composition to the subject increases Th2, Th17, and/or Tfh levels in the subject from about 350% to about 800% relative to a control subject.
10. The method of clause 9, wherein Th2 is increased by about 370%.
11. The method of clause 9, wherein Th17 is increased by about 760%.
12. The method of clause 9, wherein Tfh is increased by about 600%.
13. The method of any preceding clause, wherein administration of the composition to the subject produces an increase in serum IL-2 levels in the subject relative to a control subject.
14. The method of clause 13, wherein administration of the composition to the subject produces an increase in serum IL-2 levels in the subject from about 10% to about 30% relative to a control subject.
15. The method of clause 1, wherein administration of the composition to the subject produces a decrease in serum Th1 and/or iTreg levels in the subject relative to a control subject.
16. The method of clause 15, wherein Th1 and/or iTreg levels are decreased from about 20% to about 65%.
17. The method of clause 16, wherein Th1 is decreased by about 30%.
18. The method of clause 16, wherein iTreg is decreased by about 60%.
19. A nutritional supplement for improving immune function and/or inhibiting inflammation comprising from about 2 mg to about 800 mg paraxanthine, either natural or synthetic.
20. The of clause 19 wherein the paraxanthine is present in amount from about 20 mg to about 600 mg.
21. The nutritional supplement of clause 19 wherein the paraxanthine is present in amount from about 50 mg to about 400 mg.
22. The nutritional supplement of any of clauses 19-21, further comprising dileucine.
23. The nutritional supplement of any of clauses 19-22, wherein the nutritional supplement is a dietary supplement.
24. The nutritional supplement of clause 23, wherein the dietary supplement is powder or a capsule.
25. The nutritional supplement any of clauses 19-22, wherein the nutritional supplement is a functional food.
26. The nutritional supplement of clause 25, wherein the functional food is a beverage, nutrition bar, yoghurt, or cereal.
27. The nutritional supplement any of clauses 19-22, further comprises one or more compounds selected from the list consisting of: aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.
28. The nutritional supplement of any of clauses 19-27, wherein the paraxanthine is derived from a natural source.
29. The nutritional supplement of any of clauses 19-27, wherein the paraxanthine is synthetic.
30. The nutritional supplement of any of clauses 19-27 further comprising one or more compounds selected from a list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.
31. A method of inhibiting inflammation in a subject in need thereof comprising administering to the subject a composition comprising about 2 mg to about 800 mg of paraxanthine.
32. The method of clause 31, wherein paraxanthine is present in the composition in amount from about 20 mg to about 600 mg.
33. The method of clause 2, wherein paraxanthine is present in the composition in amount from about 50 mg to about 400 mg.
34. The method of any of clauses 31-33, wherein the composition further comprises an effective amount of dileucine.
35. The method of any of clauses 31-34, wherein the subject has been diagnosed with an inflammatory disease or condition.
36. The method of any of clauses 31-34, wherein the subject is at risk of developing inflammatory disease or condition.
37. The method of any of clauses 31-34, wherein the subject has been diagnosed with diabetes, Crohn's disease, rheumatoid arthritis, fibromyalgia, systemic lupus erythematosus, glomerulonephritis, scleroderma, or multiple sclerosis.
38. A method of treating an inflammatory disease or condition in a subject in need thereof comprising administering to the subject a composition comprising about 2 mg to about 800 mg of paraxanthine.
39. The method of clause 38, wherein paraxanthine is present in the composition in amount from about 20 mg to about 600 mg.
40. The method of clause 38, wherein paraxanthine is present in the composition in amount from about 50 mg to about 400 mg.
41. The method of clause 40, wherein the inflammatory disease or condition is diabetes, Crohn's disease, rheumatoid arthritis, fibromyalgia, systemic lupus erythematosus, glomerulonephritis, scleroderma, or multiple sclerosis.
42. The method of any of clauses 38-41, wherein the composition further comprises second anti-inflammatory agent.
43. The method of clause 42, wherein the second anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.
44. The method of clause 41, wherein the second anti-inflammatory agent is selected from aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.
45. A method for improving joint health in a subject in need thereof comprising:
   providing the subject with a composition comprising about 25 mg to about 800 mg of paraxanthine.
46. The method of clause 45, wherein paraxanthine is present in the composition in amount from about 20 mg to about 600 mg.
47. The method of clause 45, wherein paraxanthine is present in the composition in amount from about 50 mg to about 400 mg.
48. The method of any of clauses 45-47, wherein the composition further comprises an effective amount of dileucine.
49. The method of any of clauses 45-48, wherein the subject has been diagnosed with an inflammatory joint disease or condition.
50. The method of any of clauses 45-48, wherein the subject is at risk of developing inflammatory joint disease or condition.
51. A method of treating an inflammatory joint disease comprising: administering to the subject a composition comprising about 25 mg to about 800 mg of paraxanthine.
52. The method of clause 51, wherein paraxanthine is present in the composition in amount from about 20 mg to about 600 mg.
53. The method of an of clauses 51-52, wherein paraxanthine is present in the composition in amount from about 50 mg to about 400 mg.
54. The method of any of clauses 51-53, wherein the composition further comprises an effective amount of dileucine.
55. The method of any of clauses 51-54, wherein the composition is administered in a therapeutically effective amount.
56. The method of any of clauses 51-54, wherein the composition is administered in a prophylactically effective amount.
57. The method of any of clauses 31-56, wherein administration of the composition to the subject produces a decrease in serum IL-6 levels in the subject relative to a control subject.
58. The method of clause 57, wherein administration of the composition to the subject produces a decrease in serum IL-6 levels in the subject from about 5% to about 15% relative to a control subject.
59. The method of clause 58, wherein administration of the composition to the subject produces a decrease in serum IL-6 levels in the subject of about 8% relative to a control subject.
60. The method of any of clauses 31-59, wherein administration of the composition to the subject produces an increase in serum IL-10 levels in the relative to a control subject.
61. The method of clause 60, wherein administration of the composition to the subject produces an increase in serum IL-10 levels in the subject from about 5% to about 20% relative to a control subject.
62. The method of clause 61, wherein administration of the composition to the subject produces an increase in serum IL-10 levels in the subject of about 8% relative to a control subject.
63. The method of any of clauses 31-62, wherein administration of the composition to the subject produces a decrease in serum CRP levels in the subject relative to a control subject.
64. The method of clause 63, wherein administration of the composition to the subject produces a decrease in serum CRP levels in the subject from about 15% to about 30% relative to a control subject.
65. The method of clause 64, wherein administration of the composition to the subject produces a decrease in serum CRP levels in the subject of about 18% relative to a control subject.
66. A method for enhancing immune function in a subject, comprising:
   providing the subject with a composition comprising about 10 mg to about 2000 mg of 1-methylxanthine.
67. The method of clause 66, wherein 1-methylxanthine is present in the composition in amount from about 20 mg to about 600 mg.
68. The method of clause 67, wherein 1-methylxanthine is present in the composition in amount from about 50 mg to about 400 mg.
69. The method of any of clauses 66-68, wherein the composition further comprises an effective amount of dileucine.
70. The method of any of clauses 66-68, wherein the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.
71. The method of any preceding clause wherein the 1-methylxanthine is derived from a natural source.
72. The method of any preceding clause wherein the 1-methylxanthine is synthetic.
73. A nutritional supplement for improving immune function and/or inhibiting inflammation comprising from about 10 mg to about 2000 mg 1-methylxanthine, either natural or synthetic.
74. The of clause 73 wherein the 1-methylxanthine is present in amount from about 20 mg to about 600 mg.
75. The nutritional supplement of clause 73 wherein the 1-methylxanthine is present in amount from about 50 mg to about 400 mg.
76. The nutritional supplement of any of clauses 73-75, further comprising dileucine.
77. The nutritional supplement of any of clauses 73-76, wherein the nutritional supplement is a dietary supplement.
78. The nutritional supplement of clause 77, wherein the dietary supplement is powder or a capsule.
79. The nutritional supplement any of clauses 73-76, wherein the nutritional supplement is a functional food.
80. The nutritional supplement of clause 79, wherein the functional food is a beverage, nutrition bar, yoghurt, or cereal.
81. The nutritional supplement any of clauses 73-76, further comprises one or more compounds selected from the list consisting of: aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.
82. The nutritional supplement of any of clauses 73-81, wherein the 1-methylxanthine is derived from a natural source.
83. The nutritional supplement of any of clauses 73-81, wherein the 1-methylxanthine is synthetic.
84. The nutritional supplement of any of clauses 73-81 further comprising one or more compounds selected from a list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.
85. A method of inhibiting inflammation in a subject in need thereof comprising administering to the subject a composition comprising about 10 mg to about 2000 mg of 1-methylxanthine.
86. The method of clause 85, wherein 1-methylxanthine is present in the composition in amount from about 20 mg to about 600 mg.
87. The method of clause 67, wherein 1-methylxanthine is present in the composition in amount from about 50 mg to about 400 mg.
88. The method of any of clauses 85-87, wherein the composition further comprises an effective amount of dileucine.
89. The method of any of clauses 85-88, wherein the subject has been diagnosed with an inflammatory disease or condition.
90. The method of any of clauses 85-88, wherein the subject is at risk of developing inflammatory disease or condition.
91. The method of any of clauses 85-88, wherein the subject has been diagnosed with diabetes, Crohn's disease, rheumatoid arthritis, fibromyalgia, systemic lupus erythematosus, glomerulonephritis, scleroderma, or multiple sclerosis.
92. A method of treating an inflammatory disease or condition in a subject in need thereof comprising administering to the subject a composition comprising about 10 mg to about 2000 mg of 1-methylxanthine.
93. The method of clause 92, wherein 1-methylxanthine is present in the composition in amount from about 20 mg to about 600 mg.
94. The method of clause 92, wherein 1-methylxanthine is present in the composition in amount from about 50 mg to about 400 mg.
95. The method of clause 94, wherein the inflammatory disease or condition is diabetes, Crohn's disease, rheumatoid arthritis, fibromyalgia, systemic lupus erythematosus, glomerulonephritis, scleroderma, or multiple sclerosis.
96. The method of any of clauses 92-95, wherein the composition further comprises second anti-inflammatory agent.
97. The method of clause 96, wherein the second anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.
98. The method of clause 95, wherein the second anti-inflammatory agent is selected from aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.
99. A method for improving joint health in a subject in need thereof comprising:
   providing the subject with a composition comprising about 25 mg to about 800 mg of 1-methylxanthine.
100. The method of clause 99, wherein 1-methylxanthine is present in the composition in amount from about 20 mg to about 600 mg.
101. The method of clause 99, wherein 1-methylxanthine is present in the composition in amount from about 50 mg to about 400 mg.
102. The method of any of clauses 99-101, wherein the composition further comprises an effective amount of dileucine.
103. The method of any of clauses 99-102, wherein the subject has been diagnosed with an inflammatory joint disease or condition.
104. The method of any of clauses 99-102, wherein the subject is at risk of developing inflammatory joint disease or condition.
105. A method of treating an inflammatory joint disease comprising: administering to the subject a composition comprising about 25 mg to about 800 mg of 1-methylxanthine.
106. The method of clause 105, wherein 1-methylxanthine is present in the composition in amount from about 20 mg to about 600 mg.
107. The method of an of clauses 105-106, wherein 1-methylxanthine is present in the composition in amount from about 50 mg to about 400 mg.
108. The method of any of clauses 105-107, wherein the composition further comprises an effective amount of dileucine.
109. The method of any of clauses 105-108, wherein the composition is administered in a therapeutically effective amount.
110. The method of any of clauses 105-108, wherein the composition is administered in a prophylactically effective amount.
111. The method of any of clauses 66-110, further comprising paraxanthine.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Methods:

Sixteen 8-week-old male Swiss Albino mice were housed in an animal room at a constant temperature (22±3° C.) and humidity (30-70%) under a 12:12 h light-dark cycle with standard laboratory diet (Purina 5L79, Rat and Mouse 18% protein; PMI Nutrition International, Brentwood, MO, USA). Distilled water was provided ad libitum. All animal experiments were reviewed and approved by the Institutional Animal Ethical Committee (IAEC) of Radiant Research Services Pvt. Ltd. (Bangalore, India). All research was conducted in accordance with the guidelines of the committee for the purpose of control and supervision of experiments on animals.

After one week of acclimation, the animals were randomly divided by body weight into two groups (n=8 per group in each test) for oral treatment once a day, at approximately the same time each day (±1 h), for 28 consecutive days: (1) vehicle control; (2) paraxanthine. The dose administered to the mice was calculated using US Food and Drug Administration for human equivalence doses (HED), assuming a human weight of 60 kg. The following HED were used in this study: 100 mg paraxanthine, (ENFINITY™, Ingenious Ingredients, L.P Lewisville, TX, USA; mouse dose: 20.5 mg/kg bw/day).

During the treatment period, exercise training was completed using a motorized treadmill (Exer 3/6, Columbus Instruments international, Columbus, OH, USA) at a moderate intensity of 20 cm/sec as maximal running speed, an incline of 10 degrees and a shock intensity of 0.2 mA, for 10 min. The speed of the treadmill was manually adjusted by increasing the belt speed by 5 cm/sec every 2 min throughout the total duration of 10 min. All animals were adapted to this procedure daily 60 min after dosing for 5 days in a week during the treatment period.

On 29th day, blood was collected from retro-orbital route for biochemical analysis. IL-2, IL-6, IL-10, assay was done by using standard ELISA assay kit method. The Tfh,/iTreg assay was done by using flow cytometry method.

### Results:

Paraxanthine overall shows strong anti-inflammatory and immune enhancing effects. Paraxanthine reduced the pro-inflammatory cytokine IL-6, increased the anti-inflammatory cytokine IL-10, and reduced C-reactive protein (CRP). CRP is a protein made by the liver which is sent into the bloodstream in response to inflammation. Reduced levels of CRP indicate reduced inflammation. Paraxanthine increased IL-2, which is linked to an increased ability to suppress intracellular infections and to kill virus infected cells.

| | IL-2 | | IL-10 | |
|---|---|---|---|---|
| | (ng/L) | IL-6 | (pg/ml) | CRP |
| | Adaptive | (pg/ml) | Anti- | (mg/L) |
| | Immunity | Pro-inflammatory | inflammatory | Inflammation |
| Control | 37.29 ± 1.88 | 369.04 ± 13 | 527.86 ± 9.22 | 0.77 ± 0.09 |
| Paraxanthine | 45.91 ± 2.68* | 340.6 ± 13.6* | 571.71 ± 16.43* | 0.64 ± 0.07* |
| Paraxanthine | 50.07 ± 1.50* | 335.6 ± 5.8* | 580.26 ± 16.43* | 0.59 ± 0.06* |
| plus | | | | |
| Dileucine | | | | |
| Delta | +23.1% | −7.7% | +8.3% | −18.3% |

| | Th1 | Th2 | Th17 | Tfh | iTreg |
|---|---|---|---|---|---|
| | (%) | (%) | (%) | (%) | (%) |
| Control | 93.54 ± 2.82 | 11.46 ± 3.16 | 3.65 ± 0.85 | 7.82 ± 1.04 | 54.47 ± 4.2 |
| Paraxanthine | 65.68 ± 3.98* | 53.63 ± 3.85* | 31.33 ± 1.31* | 54.96 ± 4.44* | 23.68 ± 2.62* |
| Delta | −29.8% | +368.0% | +758.4% | +602.8% | −56.5% |

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

What is claimed is:

1. A method of inhibiting inflammation in a subject in need thereof comprising administering to the subject a composition comprising about 2 mg to about 800 mg of paraxanthine.

2. The method of claim 1, wherein paraxanthine is present in the composition in amount from about 50 mg to about 400 mg.

3. The method of claim 1, wherein the composition further comprises an effective amount of dileucine.

4. The method of claim 1, wherein the subject has been diagnosed with an inflammatory disease or condition.

5. The method of any of claim 1, wherein the subject is at risk of developing inflammatory disease or condition.

6. The method of claim 1, wherein the subject has been diagnosed with diabetes, Crohn's disease, rheumatoid arthritis, fibromyalgia, systemic lupus erythematosus, glomerulonephritis, scleroderma, or multiple sclerosis.

7. The method of claim 1, wherein administration of the composition to the subject produces a decrease in serum IL-6 levels in the subject from about 5% to about 15% relative to a control subject.

8. The method of claim 1, wherein administration of the composition to the subject produces an increase in serum IL-10 levels in the subject from about 5% to about 20% relative to a control subject.

9. The method of claim 1, wherein administration of the composition to the subject produces a decrease in serum CRP levels in the subject from about 15% to about 30% relative to a control subject.

10. A method of treating an inflammatory disease or condition in a subject in need thereof comprising administering to the subject a composition comprising about 2 mg to about 800 mg of paraxanthine.

11. The method of claim 10, wherein the inflammatory disease or condition is diabetes, Crohn's disease, rheumatoid arthritis, fibromyalgia, systemic lupus erythematosus, glomerulonephritis, scleroderma, or multiple sclerosis.

12. The method of claim 10, wherein the composition further comprises second anti-inflammatory agent.

13. The method of claim 12, wherein the second anti-inflammatory agent exerts anti-inflammatory effects by: inhibiting prostaglandins, increasing macrophage mediated phagocytosis, suppressing cytokine-driven inflammation, inhibiting cytokines, inhibiting histone deacetylation, inhibiting kinases, stimulating PPAR and/or inhibiting proteases.

14. The method of claim 11, wherein the second anti-inflammatory agent is selected from aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones.

15. A method for enhancing immune function in a subject, comprising:
providing the subject with a composition comprising about 2 mg to about 800 mg of paraxanthine.

16. The method of claim 15, wherein paraxanthine is present in the composition in amount from about 50 mg to about 400 mg.

17. The method of claim 16, wherein administration of the composition to the subject produces an increase in serum Th2, Th17, and/or Tfh levels in the subject from about 350% to about 800% relative to a control subject.

18. The method of claim 15, wherein the composition further comprises an effective amount of dileucine.

19. The method of claim 15, wherein administration of the composition to the subject produces an increase in serum IL-2 levels in the subject from about 10% to about 30% relative to a control subject.

20. The method of claim 15, wherein the composition further comprises one or more compounds selected from the list consisting of: Cyclosporine, tacrolimus, rapamycin, Omega-3 fatty acids, Curcumin, S-adenosylmethionine, Zinc, Green tea extract, Frankincense (Boswellia serrata resin), Capsaicin, Cat's claw (uncaria plants, including Uncaria tomentosa and Uncaria guianensis), Schizonepeta tenuifolia, Pomegranate, Moringa oleifera, Ecklonia cava, Limonene, Sunifiram, Sulforaphane, Angelica gigas, Ascophyllum nodosum, Scutellaria baicalensis, celery seed extract, Sesamin, Feverfew, Taurine, Rosmarinic Acid, Evodia rutaecarpa, Green Tea Catechins, Punicalagins, Artemisia iwayomogi, Pyrroloquinoline quinone, N-Acetylcysteine, King Oyster, Methylsulfonylmethane, alpha-lipoic acid, pine pollen, Sophora flavescens, Ophiopogon japonicus, Stephania tetrandra, Crataegus pinnatifida, grape seed extract, Bladderwrack, Paederia foetida, Benfotiamine, Rubus coreanus, Punicic Acid, Sea Buckthorn, Hibiscus rosasinensis, Phellodendron amurense, Resveratrol, Quercetin, Rooibos, Olive leaf extract, Pterostilbene, Eucommia ulmoides, Diindolylmethane, Anatabine, Serrapeptase, Pelargonidin, watercress, Astaxanthin, Piceatannol, Fish Oil, Glutathione, Orthosiphon stamineus, Aronia melanocarpa, blueberry, Tripterygium wilfordii, Boerhaavia diffusa, Whey Protein, Bromelain, Panax ginseng, Aloe vera, cocoa extract, stinging nettle, garlic, Centella asiatica, Astragalus membranaceus, Dendrobium, Vitamin C, Spirulina, Berberine, Ganoderma lucidum, Vitamin C, Vitamin D, Vitamin E, lutein, leucine, dileucine, trileucine, tetraleucine Pau d'arco, AHCC, rhodiola ashwagandha, shitake, maitake, turkey tail, monolaurin, lysine, Ergothioneine, medium chain triglycerides (MCTs) and butyrate.
